(19) United States
(12) Patent Application Publication
(10) Pub. No.: US 2013/0199543 A1
(43) Pub. Date: Aug. 8, 2013

(54) STERILE DRAPE SUPPORT

(71) Applicants: E. Kathryn Hinson, Orlando, FL (US); Luis Oliden Fernandez, St. Cloud, FL (US)

(72) Inventors: E. Kathryn Hinson, Orlando, FL (US); Luis Oliden Fernandez, St. Cloud, FL (US)

(21) Appl. No.: 13/743,205

(22) Filed: Jan. 16, 2013

Related U.S. Application Data
(60) Provisional application No. 61/587,534, filed on Jan. 17, 2012.

(51) Int. Cl. A61B 19/02; A61B 19/08

(52) U.S. Cl. CPC A61B 19/0256; A61B 19/08
USPC 128/849

(57) ABSTRACT

A sterile drape support includes a clamp, a swivel mechanism coupled to the clamp, and an elongate support bar extending from a proximal end to a distal end. The proximal end of the support bar is coupled to the swivel mechanism so that the support bar is pivotable with respect to the clamp. For use during a medical procedure, the clamp is coupled to a bed, procedure table, or another object positioned near the bed or table during the procedure, such as a cart. A sterile drape or dressing is draped over the support bar so that the drape is held off the patient's face during the procedure. A method of preparing a patient for a medical procedure includes obtaining a sterile drape support, attaching the support to an object near the patient's bed or procedure table, and draping a sterile dressing over the drape support.

### CROSS-REFERENCE TO RELATED APPLICATIONS

This application claims priority benefit of U.S. Provisional Application No. 61/587,534, filed Jan. 17, 2012, the entirety of which is hereby incorporated by reference herein.

### BACKGROUND

1. Field

The present disclosure generally relates to sterile drapes used to create a sterile zone for various medical procedures and a sterile drape support for such sterile drapes.

2. Description of the Related Art

Various medical procedures require a sterile field to prevent or reduce the risk of contamination of the working area and infection for the patient. Sterile drapes are often used to create the sterile field.

Peripherally inserted central catheters (PICC or PIC lines) and midline catheters are intravenous access devices that can be used for an extended period of time, for example, for prolonged chemotherapy, antibiotic therapy, or parenteral nutrition. The catheter is inserted in a peripheral vein such as, for example, the cephalic, basilica, or brachial vein, in the patient's upper arm. It is then advanced through the vascular system toward the heart. The insertion procedure can last about an hour or longer.

PICC or midline catheter insertion is a sterile procedure, although it can be performed outside the operating room. When the insertion procedure is performed in the patient's room, a sterile field must be created and maintained throughout the procedure. This is often accomplished by placing a sterile drape over the patient.

If a full-body drape is used to create the sterile field, the drape usually covers the patient's face. This can interfere with the doctor or nurse's observation of and communication with the patient and can cause the patient to feel claustrophobic, among other things.

### SUMMARY

The sterile drape support of the present disclosure advantageously holds the sterile drape off the patient's face during a PICC line insertion or other procedure to help alleviate these and other problems. The sterile drape support can also be used for other medical procedures that require a sterile field.

In some embodiments, a sterile drape support includes a clamp, a swivel mechanism coupled to the clamp, and an elongate support bar extending from a proximal end to a distal end. The proximal end of the support bar is coupled to the swivel mechanism so that the support bar is pivotable relative to one or more axes with respect to the clamp.

In some embodiments, a sterile drape support system includes a clamp, a swivel mechanism coupled to the clamp, an elongate support bar extending from a proximal end to a distal end, and a sterile dressing. The proximal end of the support bar is coupled to the swivel mechanism so that the support bar is pivotable with respect to the clamp. In use, the clamp is coupled to an object near a bed, table, or other support surface on which a patient can lie during a medical procedure. In some embodiments, the support bar extends over at least a substantial portion of a width of the patient support surface. The sterile dressing is draped over the support bar so that the support bar holds the dressing off the patient's face during the procedure.

In some embodiments, a medical cart for use during a medical procedure includes a movable cart and a sterile drape support. The sterile drape support includes a clamp, a swivel mechanism coupled to the clamp, and an elongate support bar that extends from a proximal end to a distal end. The proximal end of the support bar is coupled to the swivel mechanism so that the support bar is pivotable with respect to the clamp. The clamp is coupled to the cart.

In some embodiments, a method of preparing a patient for a sterile medical procedure includes obtaining a sterile drape support that includes a clamp, a swivel mechanism coupled to the clamp, and an elongate support bar that extends from a proximal end to a distal end. The proximal end of the support bar is coupled to the swivel mechanism so that the support bar is pivotable with respect to the clamp. The method further includes attaching the clamp to an object near a bed, table, or other surface on which a patient can lie during the procedure and draping a sterile dressing over the support bar so that the dressing is held off the patient's face.

### BRIEF DESCRIPTION OF THE DRAWINGS

These and other features, aspects, and advantages of the present disclosure are described with reference to the drawings of certain embodiments, which are intended to schematically illustrate certain embodiments and not to limit the invention.

FIG. 1 shows a front view of an example embodiment of a sterile drape support coupled to a bed;

FIG. 2A shows a front perspective view of an attachment portion of the sterile drape support of FIG. 1;

FIG. 2B shows a front perspective view of the sterile drape support of FIG. 1;

FIG. 3 shows a front perspective view of an example embodiment of a sterile drape support coupled to a cart; and

FIG. 4 shows an example embodiment of a method of using a sterile drape support.

### DETAILED DESCRIPTION

For purposes of summarizing the disclosure and the advantages achieved over the prior art, certain objects and advantages are described herein. Of course, it is to be understood that not necessarily all such objects or advantages need to be achieved in accordance with any particular embodiment. Thus, for example, those skilled in the art will recognize that the disclosure may be embodied or carried out in a manner that achieves or optimizes one advantage or group of advantages as taught or suggested herein without necessarily achieving other objects or advantages as may be taught or suggested herein.

All of these embodiments are intended to be within the scope of the disclosure herein. These and other embodiments will become readily apparent to those skilled in the art from the following detailed description and the attached figures, the disclosure not being limited to any particular disclosed embodiment(s).

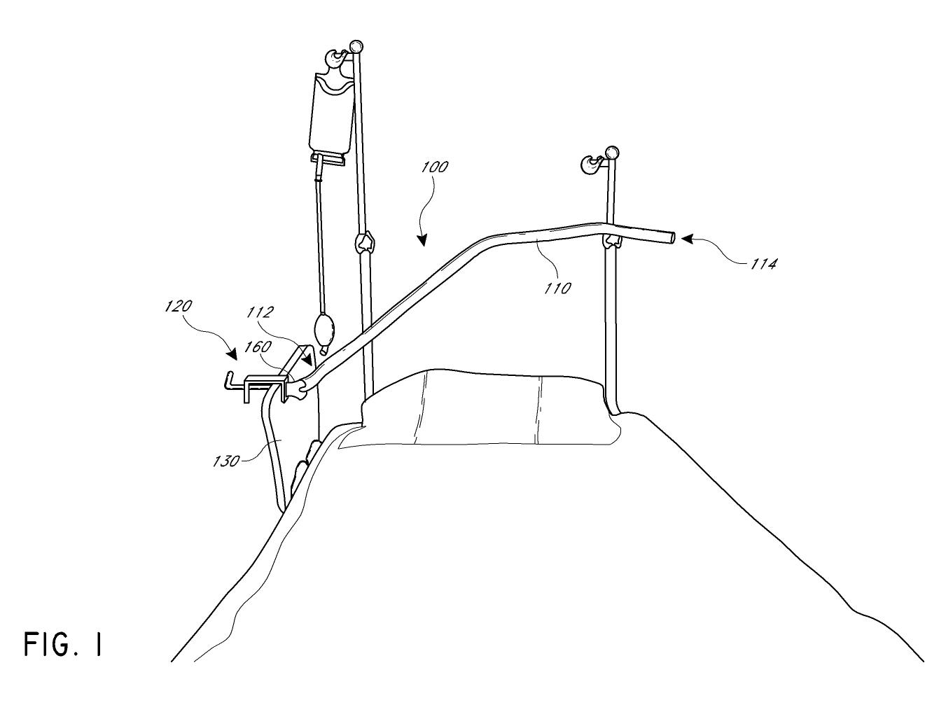

With reference to FIG. 1, an example embodiment of a sterile drape support 100 can be used, for example, for positioning a sterile drape for PICC line insertion or another sterile medical procedure. The device 100 generally includes a support bar 110 and an attachment portion 120 that allows the device 100 to be coupled to another object.

In some embodiments, the attachment portion 120 is a vise or clamp-like mechanism. For example, the attachment portion 120 can be a 3 inch C-Clamp having a width of about 3.75 inches. Other attachment mechanisms are also possible.

The support bar 110 extends from a proximal end 112 configured to be coupled to the attachment portion 120 to a distal end 114. In use, the support bar 110 extends over a surface, e.g., a bed, procedure table, etc., where a patient is positioned during the procedure. The support bar 110 can be made of a durable material or materials that can withstand cleaning and sanitization between procedures. For example, the support bar 110 can be made of PVC pipe. In some embodiments, the support bar 110 has a cap on the distal end 114 to close the distal end 114 of the pipe and cover any potentially rough surfaces. The support bar 110 can also be dressed with a probe cover during use to prevent contamination.

The device 100 can be sized and shaped so that it is functional but does not substantially interfere with medical personnel and also allows for ease of transport and storage and for use with patients in isolation. For example, in some embodiments, the support bar 110 extends far enough across the width of the bed, procedure table, or other surface to provide the desired clearance between the drape and patient's face, but does not extend substantially beyond the bed or table to avoid interfering with the medical personnel's access to the patient. In some embodiments, the support bar 110 has a length in the range of about 30 inches to about 40 inches. Shorter and longer lengths are possible for smaller or larger patients, beds, and/or procedure tables. Alternatively, an extension piece or pieces can be attached to the distal end 114 of the support bar 110 as needed to accommodate larger or wider patients, beds, and/or procedure tables. The support bar 110 can have a generally arcuate or slightly angular shape to hold the drape farther away from the patient's face during the procedure. In some embodiments, the device 100 separates the drape from the patient's face by a height of about 10 inches to about 15 inches.

In one example embodiment, the support bar 110 has a total length of about 36 inches and extends straight from the attachment portion 120 at an angle of about 45 degrees above a horizontal axis extending from the proximal end 112. The angle of the support bar 110 changes downwardly at about 19 inches from the proximal end 112 and about 28.5 inches from the proximal end 112 so that the support bar 110 approaches horizontal between about 19 inches and about 28.5 inches from proximal end 112 and angles slightly downward between about 28.5 inches from proximal end 112 and the distal end 114. The support bar 110 can create a distance between the drape and the patient's face of about 13 inches. Other sizes and configurations are also possible.

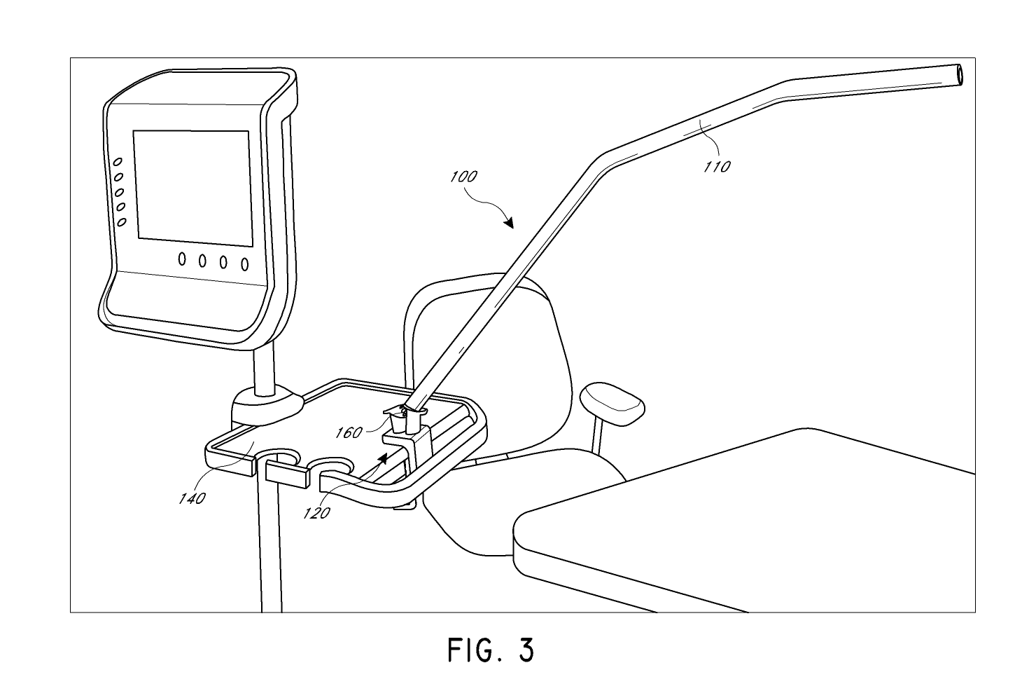

In some embodiments, the support bar 110 is movably coupled to the attachment portion 120 so that the support bar 110 and attachment portion 120 can pivot or otherwise be adjusted relative to each other. For example, the support bar 110 can be attached to a swivel mechanism 160, e.g., a ball swivel, which is attached to the attachment portion 120. The swivel mechanism 160 can allow linear and/or rotational movement of the support bar 110 and/or attachment portion 120 in one or more directions and/or about one or more axes. For example, in some embodiments, the swivel mechanism 160 allows the attachment portion 120 to be adjusted between an orientation in which an axis of actuation of the attachment mechanism is horizontal, for example as shown in FIG. 1, and an orientation in which the axis of actuation is vertical, for example as shown in FIG. 3. In some embodiments, the swivel mechanism 160 allows the attachment portion 120 to be attached to the top (as shown in FIG. 1), bottom, or any side of an object. In some embodiments, the swivel mechanism 160 allows the support bar 110 to be raised, lowered, and/or moved forward or backward relative to the attachment portion 120 and/or patient support surface. In some embodiments, the swivel mechanism 160 allows for other or alternative adjustments of the support bar 110 and/or attachment portion 120 relative to each other and/or the patient support surface.

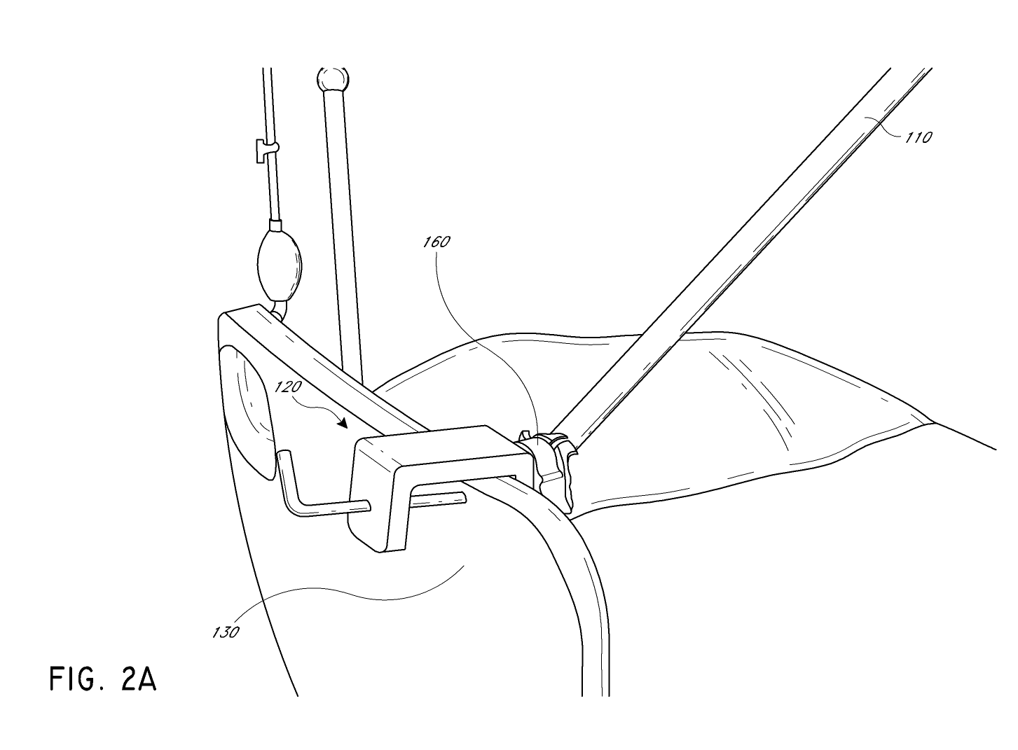

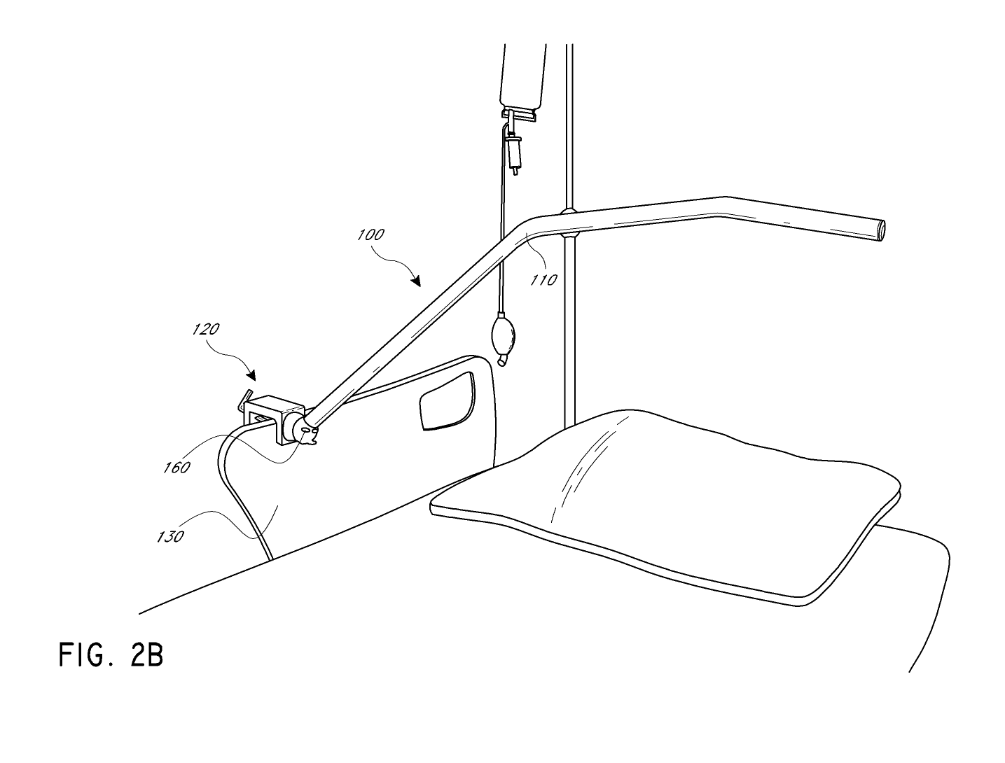

This advantageously allows the device 100 to be attached to various objects in different orientations and adjusted appropriately. For example, the device 100 can be attached to a railing 130 on the side of the patient's bed as shown in FIGS. 1-2B. In the illustrated embodiment, the attachment portion 120 is oriented horizontally to attach to the top of vertical railing 130. Alternatively, the device can be attached to other objects in the room, for example a cart 140 as shown in FIG. 3. In this case, the attachment portion 120 is oriented vertically to attach to the horizontal cart 140. Other orientations are also possible. Once in the desired position, the support bar 110 can be locked with respect to the attachment portion 120 to keep the device steady during the procedure. The swivel mechanism 160 allows for the device 100 to be attached to an object already in the room for convenience and space-saving purposes. The movable coupling also allows for the support bar 110 to be raised to a substantially vertical or other desired position for transport and storage.

In some embodiments, the components of the device 100, including the support bar 110, attachment portion 120, and swivel mechanism 160, if present, are permanently coupled so that the device 100 is a single piece and does not require assembly prior to use. Alternatively, the device 100 can be separated into various components for shipping, storage, etc.; however, minimizing the number of components can promote ease and speed of assembly for use.

In some embodiments, a kit includes device 100 and one or more sterile drapes. In some embodiments, a kit includes device 100 and one or more extension pieces that can be attached to the distal end 114 of the support bar 110 as needed to accommodate, for example, larger or wider patients, beds, and/or procedure tables. The device 100 can be included in a kit as a single piece or as multiple components assembled by the user prior to use. For example, a kit can include the support bar 110, attachment portion 120, and optionally the swivel mechanism 160 all as separate components. Alternatively, a kit can include the attachment portion 120 pre-assembled, temporarily or permanently, with the swivel mechanism 160 or the support bar 110 pre-assembled, temporarily or permanently, with the swivel mechanism 160. Other kit configurations are also possible.

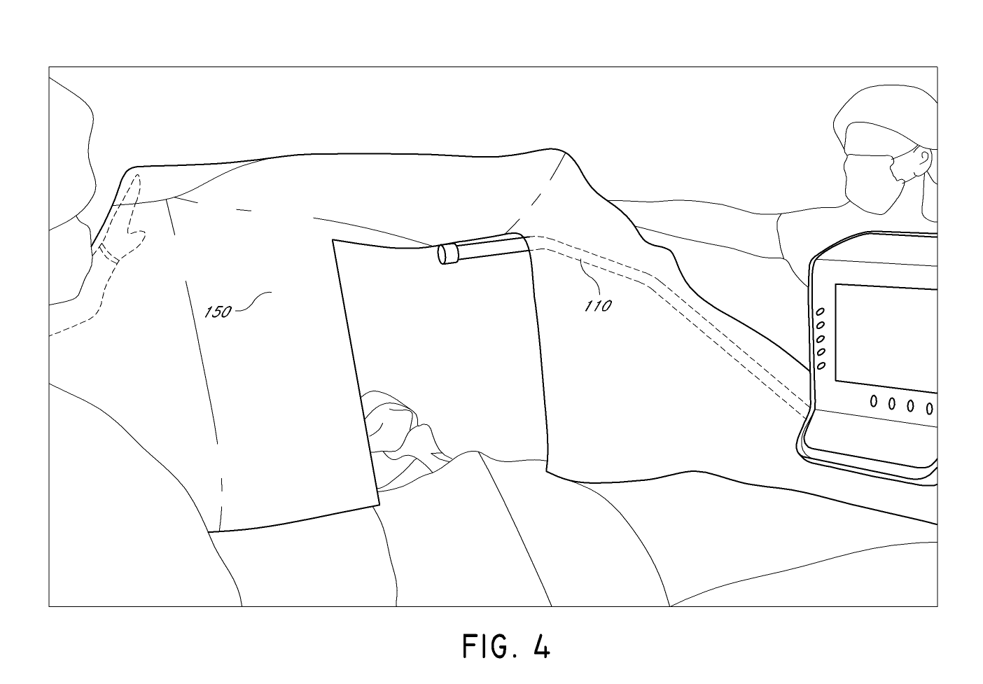

FIG. 4 shows medical professionals positioning a drape 150 over a sterile drape support bar 110 in preparation for a procedure. Once in place, the drape support 100 provides enough space between the drape 150 and the patient's face to alleviate feelings of claustrophobia and promote free ventilation during the procedure. The drape support 100 also allows the medical professionals to monitor and communicate with the patient more easily during the procedure.

Although this disclosure has been described in the context of certain embodiments and examples, it will be understood by those skilled in the art that the disclosure extends beyond the specifically disclosed embodiments to other alternative embodiments and/or uses and obvious modifications and equivalents thereof. In addition, while several variations of the embodiments of the disclosure have been shown and described in detail, other modifications, which are within the scope of this disclosure, will be readily apparent to those of skill in the art. It is also contemplated that various combinations or sub-combinations of the specific features and aspects of the embodiments may be made and still fall within the scope of the disclosure. It should be understood that various features and aspects of the disclosed embodiments can be combined with, or substituted for, one another in order to form varying modes of the embodiments of the disclosure. Thus, it is intended that the scope of the disclosure herein should not be limited by the particular embodiments described above.

What is claimed is:

1. A sterile drape support comprising:
a clamp;
a swivel mechanism coupled to the clamp; and
an elongate support bar extending from a proximal end to a distal end, the proximal end coupled to the swivel mechanism so that the support bar is pivotable with respect to the clamp.

2. The sterile drape support of claim 1, wherein the support bar is generally arcuate.

3. The sterile drape support of claim 1, wherein the support bar is angular.

4. The sterile drape support of claim 3, wherein an angle of the support bar with respect to a horizontal axis changes at a point about 19 inches from the proximal end and at a point about 28.5 inches from the proximal end.

5. The sterile drape support of claim 1, wherein the support bar comprises PVC pipe.

6. The sterile drape support of claim 5, wherein the support bar comprises a cap at the distal end.

7. The sterile drape support of claim 1, wherein the support bar has a total length of about 36 inches.

8. A sterile drape support system comprising:
a clamp;
a swivel mechanism coupled to the clamp;
an elongate support bar extending from a proximal end to a distal end, the proximal end coupled to the swivel mechanism so that the support bar is pivotable with respect to the clamp; and
a sterile dressing configured to be draped over the support bar in use;
wherein the clamp is configured to be coupled to an object positioned near a surface on which a patient can lie in a supine position and the support bar is configured to extend over the patient when the patient is lying on the surface; and
wherein the support bar is configured to hold the sterile dressing off the patient's face.

9. The sterile drape support system of claim 8, wherein the support bar creates a distance between the sterile dressing and patient's face in the range of about 10 inches to about 15 inches.

10. A medical cart for use during a medical procedure comprising:
a cart; and
a sterile drape support comprising:
a clamp;
a swivel mechanism coupled to the clamp; and
an elongate support bar extending from a proximal end to a distal end, the proximal end coupled to the swivel mechanism so that the support bar is pivotable with respect to the clamp;
wherein the clamp is coupled to the cart.

11. A method of preparing a patient for a medical procedure comprising:
obtaining a sterile drape support comprising:
a clamp;
a swivel mechanism coupled to the clamp; and
an elongate support bar extending from a proximal end to a distal end, the proximal end coupled to the swivel mechanism so that the support bar is pivotable with respect to the clamp;
attaching the clamp to an object near a surface on which the patient can lie in a supine position during the procedure; and
draping a sterile dressing over the support bar so that the dressing is held off the patient's face.
